(19) United States
(12) Patent Application Publication
(10) Pub. No.: US 2025/0132014 A1
(43) Pub. Date: Apr. 24, 2025

(54) Correlation-Driven Search Information System For Digestive Health

(71) Applicant: Johnson & Johnson Consumer Inc., Skillman, NJ (US)

(72) Inventors: Christer Bo, Fredrik SPEGEL, Helsingborg (SE); Alexander MURPHY, Zürich (CH)

(21) Appl. No.: 18/924,458

(22) Filed: Oct. 23, 2024

Related U.S. Application Data
(60) Provisional application No. 63/545,428, filed on Oct. 24, 2023.

(51) Int. Cl. G16H 20/60; G16H 40/67

(52) U.S. Cl. CPC G16H 20/60; G16H 40/67

(57) ABSTRACT

A computer system directed to digestive health, for example to diarrhea, can help direct a user to content specifically related to her experience, without requiring traditional user-entered search terms. Such a computer system may receive user-specific, timeseries data indicative of user-recorded diet, mood, and diarrhea symptoms. The computer system may capture this data in a smart phone daily diary application. The computer system may use this data to determine a correlation between the user-recorded diet and mood information and the user-recorded diarrhea symptom information. Then, an appropriate unit of content may be selected from a plurality of content units, such as from a database of digestive health information, based on the determined correlation. The selected unit can be presented to the user.

### PRIORITY CLAIM

The present application claims priority to U.S. Provisional Application Ser. No. 63/545,428 filed Oct. 24, 2023, the entire contents of which is hereby incorporated by reference herein.

### BACKGROUND

Diarrhea is a common medical condition characterized by the frequent passage of loose, watery, and often uncontrollable stools or bowel movements. It is typically associated with an increased frequency of bowel movements, and these movements may be accompanied by a sense of urgency. Diarrhea can vary in severity and duration, ranging from a mild and short-lived episode to a more persistent and severe condition.

There are many causes of diarrhea, including, for example, infections, specific food intolerances, medication side-effects, general dietary factors, chronic medical conditions, and stress.

Because the causes of diarrhea are wide and varied, obtaining accurate and helpful information through traditional methods is difficult. For example, a person suffering from diarrhea may engage a traditional Internet search engine to obtain information. Faced with a blank search engine query box, where does she even start? How does she craft a search query that will help her find information most helpful for her specific diarrhea experience and causes, especially when she may not even recognize them?

Moreover, the social stigma associated with diarrhea may dissuade sufferers from openly communicating about their condition. Diarrhea sufferers may feel uncomfortable asking for help from health care professionals, caretakers, friends, or relatives. This isolation and self-imposed silence makes it even more difficult for diarrhea sufferers to know the right language to use when engaging a traditional search engine.

Plainly, a traditional search engine is not technically suitable for finding content when the user is not equipped to prepare an adequate query.

Without access to suitable information either online or through other people, sufferers' symptoms do not improve, or may get worse. Sufferers also often report low confidence in managing their condition, which is debilitating in its own right because it leads to further isolation and reluctance to participate in activities that could otherwise be enjoyed.

### SUMMARY

As disclosed herein, a computer system directed to digestive health, for example to diarrhea, can help direct a user to content specifically related to her experience, without requiring traditional user-entered search terms. Such a computer system may receive user-specific, timeseries data indicative of user-recorded diet, mood, and diarrhea symptoms. For example, a computer system may capture this data in a smart phone daily diary application. The computer system may use this data to determine a correlation between the user-recorded diet and mood information and the user-recorded diarrhea symptom information. Then, an appropriate unit of content may be selected from a plurality of content units, such as from a database of digestive health information, based on the determined correlation. The selected unit can be presented to the user.

This query and retrieval of content can occur without requiring the user to craft words into a particular query, which is often difficult given the wide range of experiences and causes of diarrhea. And the use of user-specific, timeseries data indicative of user-recorded diet, mood, and diarrhea symptoms to drive content selection represents a significant technical improvement to traditional content delivery techniques.

In particular, methods of the present invention ensure that content units sent to the user are more closely associated with their specific user-recorded data, and thus more relevant to their unique digestive health, than previously known methods for digestive health content selection could provide.

It has been found that having had access to content units selected by methods of the present invention, users are consistently able to reduce diarrhea symptom severity and can more confidently manage their condition.

### BRIEF DESCRIPTION OF THE DRAWINGS

FIGS. 1A & B depict an example system and an example computing device, respectively.

FIG. 2 illustrates an example search engine and content delivery system based on user-specific diet, mood, and diarrhea symptom data.

FIG. 3 is a messaging diagram of an example search engine and content delivery system.

FIG. 4 is a process flow diagram illustrating an example method for selecting a content unit based on user-specific diet, mood, and diarrhea symptom data.

FIG. 5 is an example user interface for a smart phone application with diet, mood, and diarrhea symptom tracking capabilities.

FIG. 6 is an example user interface for user-specific mood and diarrhea symptom information collection.

FIGS. 7A & B are example user interfaces for user-specific diet information collection.

FIGS. 8A-D are user interfaces illustrating example, content units that are selected and delivered to the user based on user-specific diet, mood, and diarrhea symptom data.

FIG. 9 is a graph illustrating levels in user confidence in managing their digestive health over a three month period.

### DETAILED DESCRIPTION

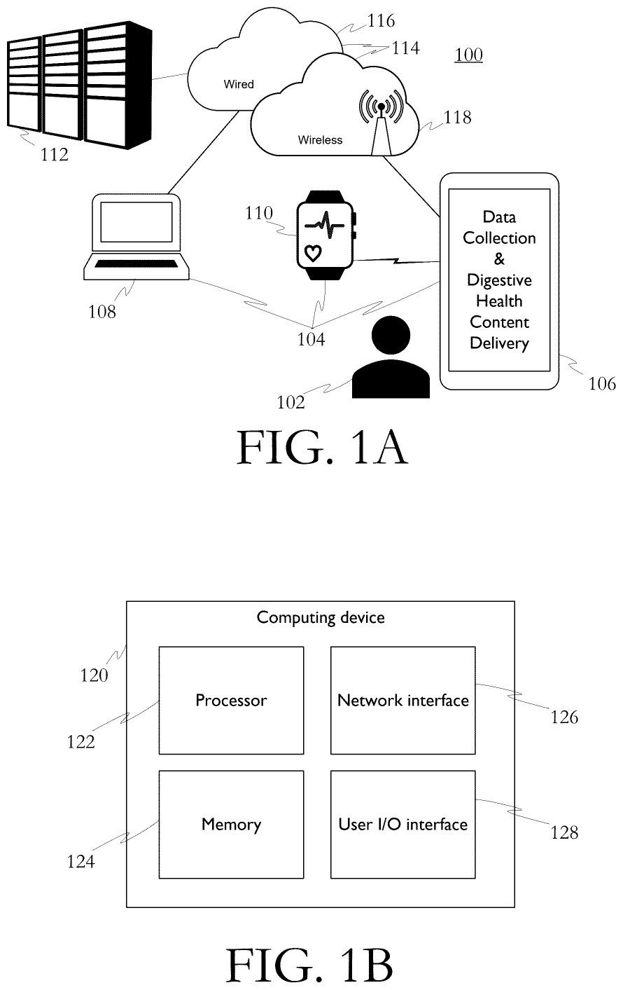

FIG. 1A depicts an example system 100. A user 102 may operate a user device 104 (such as a smartphone 106, personal computer 108, smartwatch 110, or the like). The computing device may provide access to a digestive health application. The computing device may include a stand-alone digestive health application. The user device 104 may include a digestive health application that operates in concert with a corresponding application available at a computing system 112. In an example, the computing device may include an internet enabled application, such as a browser, web application, iOS App Clip, or the like, that provides access to a digestive health application program via the computing system 112. The computing system 112 may include any device suitable for processing, sending, and receiving data, such as a computer server, server system, cloud resource, virtual machine, or the like.

The computing system 112 may communicate with the user device 104 via one or more networks 114 including, for example, a wired network 116, a wireless network 118, or the like. The wired network 116 may include any computing network technology suitable for transmitting and receive data terrestrially, including, for example, the Internet, private networks, virtual private networks, leased data facilities, internet service provider access networks, and the like. The wireless network 118 may include any computer network technology suitable for transmitting and receiving data wirelessly, such as WiFi, 802.11-based networks, cellular networks such as 3G, 4G, 5G networks, or the like. The wired network 116 and the wireless network 118 may be interconnected.

The user 102 may use the user device 104 to track daily mood, diet, and digestive health symptoms, such as diarrhea, for example. The computing system 112 may provide to the user 102 via the user device 104 appropriate content selected based on the user's uploaded data. The user device 104 may include any computing system suitable for processing and communicating data. For example, the user device may include a smartphone 106, tablet, personal computer 108, smartwatch 110, smart appliance, digital audio assistant, or the like.

In an example, an application on a smartphone 106 may include a digestive health application. The application on the smartphone 106 may include a native application, a web application, an iOS AppClip, etc. The application may operate in a standalone capacity and/or may operate in connection with networked computing resources, such a computing system 112. In an example, the smartphone 106 may include a browser application that delivers the digestive health application experience to the user 102 via interaction with a digestive health application on the computing system 112. The computing system 112 may include one or more servers configured to inter-operate with the digestive health application and/or deliver the digestive health application.

In an example, a user 102 may access the digestive health app via a personal computer 108. The personal computer may be connected to the wired network 116 via an internet service provider, for example. The application may operate in a standalone capacity and/or may operate in connection with networked computing resources, such a computing system 112. In an example, the personal computer 108 may include a browser application that delivers the digestive health application experience to the user 102 via interaction with a digestive health application on the computing system 112.

The computing system 112 resources may include any computing technology suitable for storing, processing, transmitting, and receiving data. For example, the computing system 112 may include a computer server, such as web server, blade server. The computing system 112 may include cloud computing resources such as a virtual machine, for example.

In an example, the user device 104 may interact with the computing system 112 to deliver a digestive health application experience to the user 102. For example, the computing system 112 may interact with the user device 104 via one or more Application Programming Interface (API) calls, delivered, for example, via one or more Hypertext Transfer Protocol (HTTP) messages (e.g., GET, PUSH, POST, and the like).

In an example, an application on a smartphone 106 may communicate with an application on a smartwatch 110 via a wireless protocol such as Bluetooth, Bluetooth low energy, or the like. The smartwatch 110 and smartphone 106 may enable the user to access the digestive health application. The smartwatch 110 and/or smartphone 106 may access networked computing resources, such as the computing system 112, via one or more networks 114, such as a wireless network 118 and/or a wired network 116.

FIG. 1B details an example computing device 120. The architecture of the computing device 120 is suitable for any of the computers disclosed herein, including for example, the user device 104, smartphone 106, personal computer 108, smartwatch 110, computing system 112, and the like. The computing device 120 may include a processor 122,

As shown in figure two, an example computing user device may include a processor, a memory, and everything else that it might include. Also, for example, a computing resource may include real or virtual hardware, including a processor 122, a memory 124, a network interface 126, a user input-output (I/O) interface 128, and the like.

The processor 122 may be a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Arrays (FPGAs) circuits, any other type of integrated circuit (IC), a state machine, and the like. The processor 122 may be configured, for example configured with application software via computer readable code, to perform the functions of the computers disclosed herein, including for example, the user device 104, smartphone 106, personal computer 108, smartwatch 110, computing system 112, and the like. For example, the processor 122 may be configured to perform any of the computer methods disclosed herein.

The memory 124 may include any computing hardware suitable for storing information in connection with the processor 122. The processor 122 may access information from, and store data in, in the memory 124. The memory 124 may include any type of suitable hardware, such as the non-removable memory and/or the removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. In an example, the processor 122 may access information from, and store data in, memory 124 that is not physically located on the computing device 120, such as via virtual storage resources, storage server, Network Access Server (NAS), and the like.

The network interface 126 may include any computing hardware suitable for the transmission and reception of data over a computer network. For example, the network interface 126 may include hardware such as an Ethernet network adapter, a Wi-Fi network adapter, a Bluetooth adapter, a USB network adapter, a fiber optic network adapter, a cellular network adapter, and the like. The network interface 126 may enable communication between devices via networks 114, such as wired network 116 and/or a wireless network 118. The network interface 126 may enable communication between a smartwatch 110 and a smartphone 106.

The user I/O interface may provide an interface between the computing device 120 and the user 102. The user I/O interface 128 may include any hardware suitable for communicating information between the computing device 120 and the user 102. For example, the user I/O interface 128 may include hardware such as a speaker/microphone, a physical keypad, a virtual keypad, a display, a touch display, a mouse, indicators lights, and the like. For example, the smartphone 106 may include a touch display enabling the user 102 to interact with a digital health application, to receive information from the user 102 and to provide resultant content to the user 102.

The computing device 120 may include other peripherals, which may include one or more software and/or hardware modules that provide additional features, functionality and/or wired or wireless connectivity. For example, the peripherals may include an accelerometer, an e-compass, a satellite transceiver, a Global Positioning System (GPS) receiver, a digital camera (for photographs and/or video), a universal serial bus (USB) port, a vibration device, a television transceiver, a hands free headset, a Bluetooth® module, a frequency modulated (FM) radio unit, a digital music player, a media player, a video game player module, an Internet browser, a Virtual Reality and/or Augmented Reality (VR/AR) device, an activity tracker, and the like. The peripherals may include one or more sensors, the sensors may be one or more of a gyroscope, an accelerometer, a hall effect sensor, a magnetometer, an orientation sensor, a proximity sensor, a temperature sensor, a time sensor; a geolocation sensor; an altimeter, a light sensor, a touch sensor, a magnetometer, a barometer, a gesture sensor, a biometric sensor, and/or a humidity sensor.

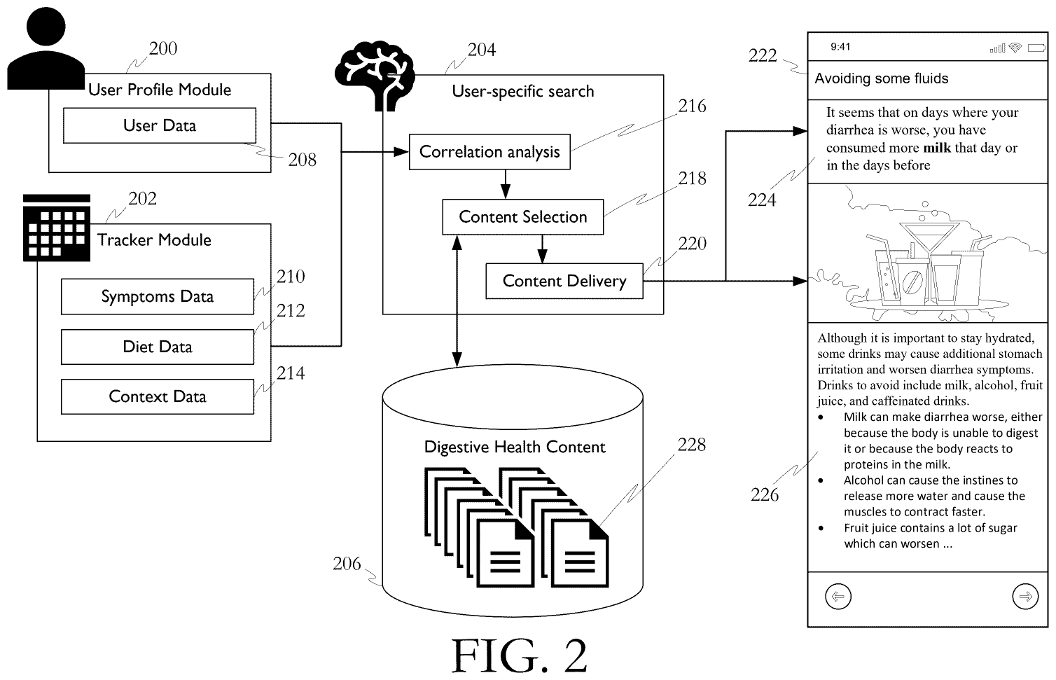

FIG. 2 illustrates an example search engine and content delivery system based on user-specific diet, mood, and diarrhea symptom data. The block diagram illustrates the operation of a digestive health app. The application structure may include functional elements such as a user profile module 200, a tracker module 202, a user-specific search module 204, and digestive health content datastore 206, for example. The application may be developed using any suitable application development technology, including, for example, Xcode with Swift, Objective-C, Android Studio with Java and/or Kotlin, React (e.g., a framework that uses JavaScript to build native-like apps for both iOS and Android), Flutter (e.g., a framework that uses the Dart programming language), Xamarin (e.g., a cross-platform development framework using C# and .NET), Ionic (e.g., a framework built on top of Angular and Apache Cordova), responsive design technology (e.g., using Hypertext Markup Language (HTML), Cascading Style Sheets (CSS), and/or JavaScript to adapt to different screen sizes and devices), Progressive Web App technology (e.g., apps that offer a native app-like experience, are accessed through web browsers on smartphones, can work offline, and be installed on the home screen), no-code platforms (e.g., Bubble, Adalo, and/or OutSystems), and the like.

The user profile module 200 may include functionality and data associated with the user of the digestive health application. The user profile module 200 may include user data 208. The user profile module 200 may include application code that when executed causes the application to collect user data 208 from the user. For example, the user profile module 200 may cause the presentation of one or more screens to collect information about the user. For example, the screens may collect basic user information, e.g., user data 208, such as name, email address, username, display name, avatar image, date of birth, weight, sex, location, and the like. For example, the user data 208 may be used for user registration with the digestive health app. The user data 208 may include data from each user of the application.

The user profile module 200 may collect user data 208 relevant to the user's digestive health. For example, the user profile module 200 may collect user data 208 entered by the user responsive to a digestive health survey. The survey may include questions such as the following: How often do you experience diarrhea? Have you had diarrhea in the last month? Have you had diarrhea in the last week? Do you have other digestive ailments? Have you had symptoms in the last month, where the symptoms include selectable items such as abdominal pain, cramps, bloating, wind, nausea, and the like)? What kinds of foods do you cat? How would you describe the frequency of the symptoms experienced (e.g., none, rarely, sometimes, often, very often)? How do you currently manage your digestive health symptoms? How confident are you in managing your digestive health? The data format for the user's answers may include ratings (e.g., a number selected by the user between 1 and 10), a single select (e.g., user selects a single item from a presented list), and/or a multi-select (e.g., user selects multiple items from a list). The user profile module 200 may include functionality to convert the information collected in the user data 208 to establish a baseline digestive health rating for the user. The rating may use any algorithm appropriate for reducing the general digestive health of the user to a metric. For example, the rating may be determined by setting a point-value for each available answer in the survey, summing the point-values for the selected answers, and normalizing the total on a 1-to-10 scale. The rating may be scaled for biographical information such as age, sex, and the like. The user profile module 200 may cause the survey to be presented in segments to the user, each separated by days (such a timing may prevent the user from being overwhelmed). The user profile module 200 may cause the survey to presented subsequent times to the user over the user's duration with the application to obtain an updated rating and assess a user's progress. In an example, the user's rating may be presented to the user. In an example, the user profile module 200 may present to the user a synoptic representation of the user-entered data. The synoptic representation may include a “snapshot” view of the user's independent self rating information. Such a synoptic representation may remind the user of their journey with digestive health and may improve adherence with the application.

The user data 208 may include goal information entered by the user. The user may record one or more goals for their digestive health. The application may present these goals back to the user over time to provide further engagement with the application, encouragement to the user, and for assessing the user's progress. The goal may include a rating target.

The tracker module 202 may include data and functionality associated with tracking a user's daily experience with digestive health. For example, the tracker module may include code that, when executed, presents the user with a number of screens to receive from the user information about symptoms, mood, diet, and other digestive health related contextual information. The information from the user may include symptom data 210, diet data 212, and context data 214, which may include information related to other factors collected from the user, such as mood, and/or factors collected from other sources, such as location, weather, health tracker information (e.g., information via Apple HealthKit or other user health information data sources), and the like.

The information from the user may include time-series data. For example, the symptom data 210 may include information related to a particular symptom experience and a time/date associated with the symptom experience. Likewise, the diet data 212 may include information related to a particular food item ingested by the user and a time/date associated with the ingestion of the food item. And similarly for the context data 214, it may include, for example, a mood event from the user and a time/date associated with the mood event. The captured events and timeseries information may enable the determination of correlations in the received data between symptoms and other recorded events, such as determination of correlations between certain foods and diarrhea, for example.

The time-series data may be captured via a mechanism suitable for tracking the time/date of digitally captured events. For example, the tracker module 202 may present a graphical daily diary to the user for collecting information. For example, the tracker module 202 may include a graphical interface element, such as a button for example, that when clicked records an event and a time/date the button was clicked. For example, the tracker module 202 may include a time/date field, such as a date/time picker graphical element, for the user to enter a date/time associated with a particular event, such as a symptom, food item, mood, and/or other event.

The symptom data 210 may include any information related to digestive health symptom events. The symptom data 210 may include information related to the user experiencing a digestive health symptom event, such as a particular instance of a symptom. The digestive health symptom event may include events such as diarrhea (e.g., loose bowel movement), nausea (e.g., a feeling of queasiness and/or the urge to vomit), abdominal pain (e.g., general discomfort in the abdominal area), cramps (e.g., acute sharp and/or dull pain and/or painful tightness in the abdominal area), wind (e.g., flatulence, release of gas from the digestive tract through the anus), eructation (e.g., release of gas from the stomach through the mouth), bloating (e.g., a sensation of fullness and tightness in the abdomen, often with visible swelling), and the like. The digestive health symptom event may include events such as heartburn (e.g., burning sensation in the chest, caused by stomach acid backing up into the esophagus), constipation (e.g., difficulty passing stool, often resulting in infrequent or hard bowel movements), vomiting (e.g., expulsion of stomach contents through the mouth), blood in stool (e.g., the presence of blood in the feces, which can be bright red or dark in color), changes in stool color or consistency (e.g., unusual stool color, such as pale, black, or the like, and/or unusual stool consistency, such as greasy, clay-like, or the like), foul-smelling stool (e.g., abnormally strong and/or unpleasant odors from bowel movements), mucus in stool (e.g., the presence of mucus-like substance in the feces), loss of appetite (e.g., reduced desire to cat, sometimes, even when hungry), weight loss (e.g., unintentional and/or rapid weight loss without dieting or exercise), acid reflux (e.g., the backward flow of stomach acid into the esophagus, often causing a burning sensation), regurgitation (e.g., the involuntary flow of stomach contents into the mouth, often accompanied by a sour or bitter taste), and the like.

Symptom data 210 may be recorded in any method suitable for tracking the presence and/or the severity of a symptom event. For example, the tracker module 202 may present to the user an interface for the user to indicate symptoms in a binary fashion (whether the event was present or not), with a frequency metric, with a severity rating (e.g., on a rating scale of 1-10), and the like.

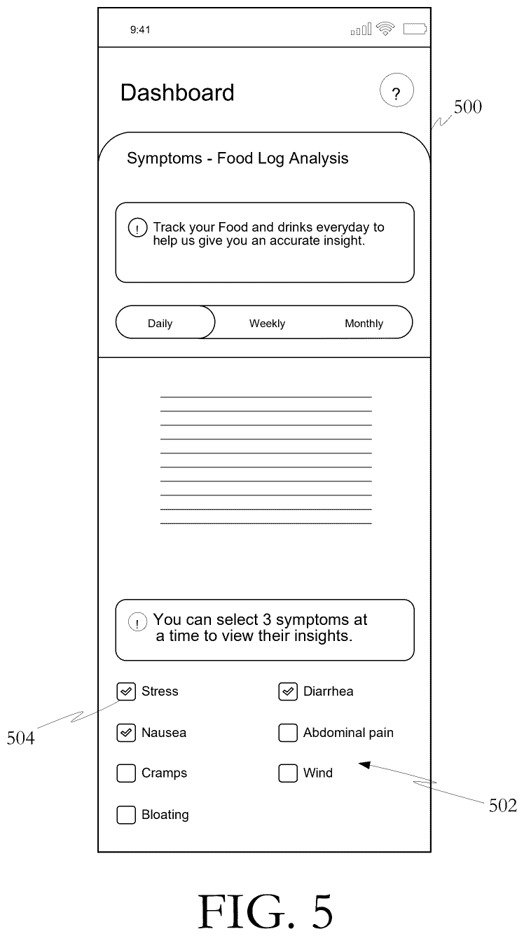

To illustrate, the tracker module 202 may present a dashboard 500, as shown in FIG. 5, to collect symptom data. For example, the dashboard 500 may include one or more check box and/or radio button graphical elements 502 to indicate symptoms. Also to illustrate, the tracker module 202 may present a daily diary 600 to capture user data, such as symptom, mood, and/or diet information. The daily diary 600 may provide one or more check box and/or radio button graphical elements 602 to indicate symptoms. The daily diary 600 may include one or more slider graphical elements 604 to indicate severity. The daily diary 600 may include a calendar graphical element 606 for the user to select a date (and/or a time, in some examples) associated with the indicated symptom, mood, and/or diet information.

The diet data 212 may include any information related to the user's ingesting of food and/or drink. For example, the diet data 212 may include any structured data suitable for characterizing food and/or drink. For example, the diet data 212 may include a listing of specific foods, categories of foods, and/or the like. For example, the diet data 212 may include information that characterizes ingested foods and/or drink. Such diet characterization information may characterize diet items by nutritional content (e.g., macronutrients such as energy content including carbohydrates, proteins, and fats; micronutrients, such as vitamins and minerals; fiber; and the like), by food group (e.g., fruits, vegetables, grains and cereals, meat and poultry, dairy products, seafood, legumes and nuts, and the like), by food origin (e.g., plant-based foods, animal-based foods, and the like), by type of food processing (e.g., fresh, raw, unprocessed and/or minimally processed foods; processed foods that have undergone some form of processing, like drying, freezing, cooking, etc.; ultra-processed foods that contain additives, preservatives, and artificial ingredients), by dietary restriction (e.g., vegetarian, lacto-vegetarian, ovo-vegetarian, vegan, halal, kosher, and the like), by culinary use (e.g., main courses, side dishes, condiments, seasonings, and the like), by meal type (e.g., breakfast foods, snacks, desserts, lunch foods, dinner foods, and the like), by cultural and/or regional categorizations (e.g., ethnic foods that may be specific to particular cultures or cuisines, regional cuisine that may be associated with specific geographic regions, and the like), by dietary health categories (e.g., gluten-free foods, low-carb foods, low-fat foods, and the like), by allergen content (e.g., indicating foods that are known allergens, such as peanuts, tree nuts, milk, eggs, soy, wheat, fish, and shellfish, and the like), by spiciness (e.g., an indication of food with the sensory perception is primarily associated with the presence of compounds like capsaicinoids, which are naturally occurring chemicals found in chili peppers and other spicy foods), and the like. Spiciness may be further characterized via a subjective heat sensation, a subjective burning and/or tingling, an objective scale (e.g., Scoville heat scale), by food/drink source (e.g., restaurant, grocery store, and the like, and regarding water, characterizations such as municipal water, bottled water, filtered water, etc.), and the like.

The diet data 212 may be recorded via any sub-system suitable for collecting diet information from a user. For example, the tracker module 202 may present a diary for collecting information about the user's diet. For example, the user may be presented with a screen requesting information about what the user has recently eaten. For example, the screen may present one or more sub-screens for breakfast, lunch, dinner, snacks, and or the like. For each sub-screen, the user may enter a food item that the user has consumed. For example, the user might have consumed eggs and bacon and home fries for breakfast and may enter information related to the identity and quantity of each component of their breakfast into the sub-screen. The interface may collect additional information related to the diet items, such as any characterization of the food items as discussed above.

In an example, tracker module 202 may include an interaction with a food database to resolve user-entered food items to their relevant characterizations (e.g., any of the characterizations discussed above). For example, the tracker module 202 may include an interaction with a third-party API (application programming interface) database. For example, the user-entered food item may be resolved into nutritionally relevant components. For example, the user-entered food item may be resolved into nutritionally relevant components by sending the user-entered food item to a third-party API database and receiving from the database the nutritionally relevant components. To illustrate, a tracker module 202 receiving a user-entered food item of an egg may interact with a food database to return and/or store in the diet data 212 the caloric, carbohydrate, fat, sodium, etc. content of the egg. The food database may have indications with relation to certain foods with relevance to digestive health, in particular diarrhea for example. The application may ask the user to indicate if the eggs were seasoned to be spicy or not, or if they were served fully cooked, or partially cooked, for example.

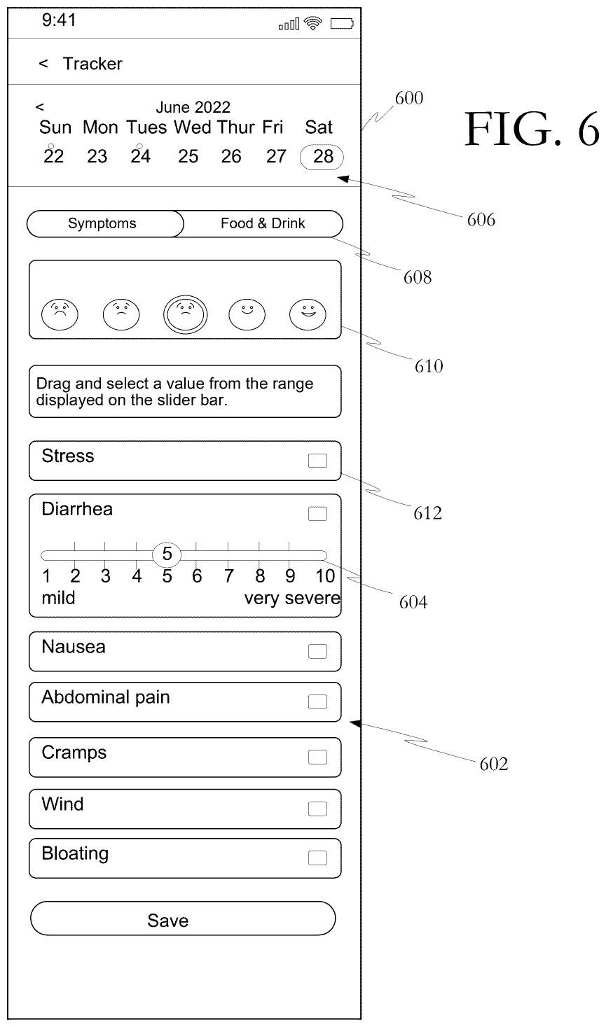

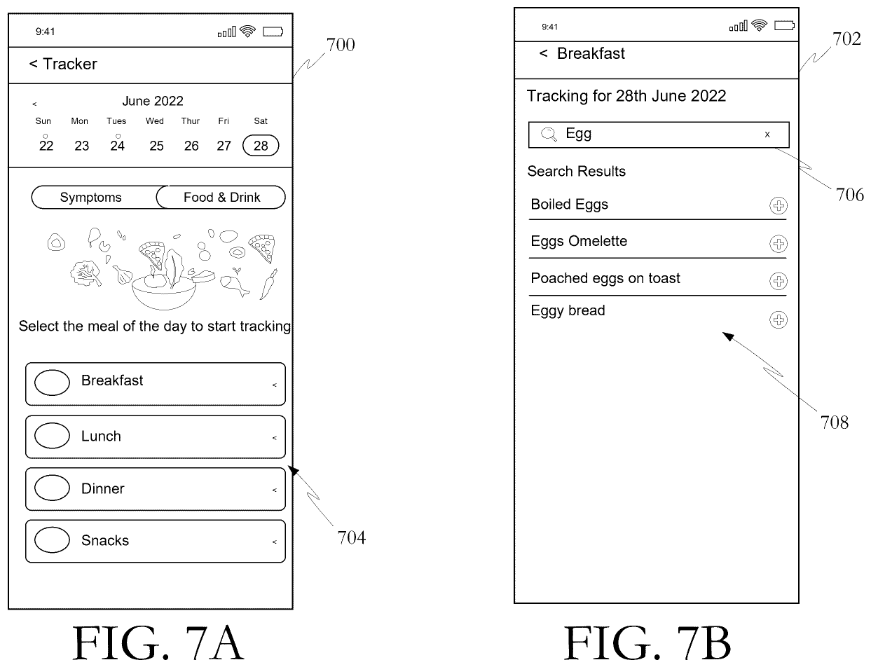

To illustrate, as shown in FIG. 6, the user may be presented with a daily diary 600 with a selector 608, to select graphical elements associated with symptoms and/or mood or to select graphical elements associated with diet corresponding to the selected date/time (e.g., via calendar graphical element 606). Selecting diet here, the user may be presented with a diet screens 700, 702, like that illustrated in FIGS. 7A & B. The diet screen 700 may receive characterization and/or refined time data from the user. As illustrated, the diet screen 700 may include on or more advance elements 704 indicating meal type. Responsive to such as selection, the diet screen 702 may include a text field 706 for entering and/or searching food items. For example, a food item entered in the text field 706 may be used to interact with a food database. Corresponding results 708 may be presented to the user for selection. Other screens may be presented to the user to enter quantity of the food item and/or any characterization information, as discussed above. In an example, the characterization information may be returned from the food database.

The context data 214 may include any other data relevant to tracking the user's digestive health, including for example user's mood. For example, the context data 214 may include life activity and/or life characterizing elements such as travel, meal location (e.g., at home, sit-down restaurant, fast food restaurant), sleep, exercise, stress, mood, weather, and the like.

The context data 214 may include information collected from the user (e.g., via one or more graphical elements). For example, the context data 214 may include the user's response to a directly posed question on a screen, such as “would you rate your mood today to be good or better?”. The context data 214 may include the user's response to a selectable element and/or a severity scale, such as selecting a stress experience and/or rating the stress experience on a scale of 1-10. The context data 214 may include information collected via an API and/or data-sharing connection with one or more other applications on the smartphone, such as via the Apple Health toolkit, for example.

Regarding mood information, the context data 214 may include information collected by any known technique for establishing a person's mood, including stress. For example, the context data 214 may include an indication from the user indicative of a person's emotional state and/or disposition at a particular time. For example, the context data 214 may include an indication from the user indicative of their emotional state and/or disposition, such as happy, sad, angry, anxious, calm, excited, depressed (e.g., a feeling of deep and/or persistent sadness, often accompanied by low energy and lack of interest), energetic, stressed (e.g., a tense feeling and/or that of feeling “on-edge” or “tightly wound”), irritable, content, hopeful, frustrated, relieved, bored, confident, nervous (e.g., an experience of unease or apprehension, perhaps related to uncertainty or anticipation), lethargic, optimistic, pensive, gloomy, elated, melancholic, resigned, illuminated, and/or the like.

The context data 214 may include information related mood/stress assessment tools. For example, the context data 214 may include information related to a mood assessment tool, such as the Beck Depression Inventory (BDI); Patient Health Questionnaire-9 (PHQ-9), which screens for symptoms of depression and assesses their severity; the Profile of Mood States (POMS); and the like. For example, the context data 214 may include information related to a stress assessment tool, such as the Perceived Stress Scale (PSS), the Holmes and Rahe Stress Scale, the Daily Hassles Scale, which measures the frequency and/or perceived stress of daily life hassles, the Trier Social Stress Test (TSST), the State-Trait Anxiety Inventory for Adults (STAI-AD), Cohen's Perceived Stress Scale, the Stress Appraisal Measure (SAM), and the like.

To illustrate, the dashboard 500, as shown in FIG. 5, may include a mood check box 504 for the user to indicate presence of stress on a particular date and/or time. To further illustrate, as shown in FIG. 6, the user may be presented with the daily diary 600 which may include one or more graphical elements for a user to indicate mood, including stress. For example, the daily diary 600 may include a graphical icon selector 610 with icons representing a range of moods (e.g., five faces of various degree of smiles) that the user can select. For example, the daily diary 600 may include a mood check box 612 (e.g., including stress) that when selected may present the user with a slider (not shown) to indicate mood severity. Such as slider (not shown) may operate like slider 604 docs.

Information from the user profile module 200 (e.g., user data 208) and/or information from the tracker module 202 (e.g., symptom data 210, diet data 212, and/or context data 214) may be used by the user-specific search module 204 for processing. For example, the user-specific search module 204 may perform correlation analysis 216, content selection 218, and/or content delivery 220. For example, information from the user profile module 200 and/or the tracker module 202 may be used to identify correlations in diet, mood, and/or other factor (e.g., recorded information) with the onset, frequency, and/or severity of symptoms, such as diarrhea. Such correlations may be used to select and/or deliver corresponding content to the user. For example, the user-specific search module 204 may select and/or deliver a content unit 222 to the user based on the correlation analysis 216. The content unit 222 may include primary content 224. For example, the primary content 224 may include a human readable summary of the user-recorded diet and mood information that represents the source of the correlation. To illustrate, if a correlation is found between milk ingestion and high diarrhea severity, the primary content may state, “it seems that on days where your diarrhea is worse, you have consumed more milk that day or in the days before.” The content unit 222 may include secondary content 226 for example, the secondary content 226 may include content selected from a plurality of content units 228, such as a plurality of content units 228 stored in the digestive health content datastore 206. Such secondary content 226 may represent content relevant to the user, e.g., content relevant in light of the correlation analysis. And to illustrate, if a correlation is found between milk ingestion and high diarrhea severity, secondary content 226 including an educational article about the relationship of various fluids, particularly including milk, may be presented to the user.

The correlation analysis 216 may include identifying one or more factors associated with the user's symptoms. For example, the correlation analysis 216 may include identifying one or more factors from the diet data 212 (including data as entered by the user and/or as food characteristics resolved from the user entered data) and context data 214 timely associated with the onset, severity, and/or frequency of a user's recorded diarrhea events in the symptom data 210. Generally, diarrhea is a condition characterized by frequent, loose, and watery bowel movements. It is a common digestive problem that can be caused by various factors, including infections, dietary issues, medications, and underlying health conditions. For example, diarrhea may be caused by infections (e.g., bacterial, viral, or parasitic infections, such as gastroenteritis), by food poisoning, by dietary factors, by medications, by digestive disorders (e.g., conditions like irritable bowel syndrome (IBS), Crohn's disease, and celiac disease can cause chronic diarrhea), by travel (e.g., diarrhea may be triggered due to changes in water and food sources), by stress and/or anxiety (e.g., emotional stress can sometimes lead to diarrhea), and by other triggers. These triggers may precede the onset and/or exacerbation of symptom by days, weeks, or months. Because of the wide-range of causes and the latency between cause and symptom, it is often difficult for a diarrhea sufferer to identify the particular causes for that individual's diarrhea. This makes it incredibly difficult for such a user to obtain content relevant to their situation. The correlation analysis 216 may algorithmically identify one or more triggers (e.g., data elements) from diet data 212 and/or the context data 214 that are correlated with symptom events from the symptom data 210.

The correlation analysis 216 may map various diet data elements, mood data elements, and/or other contextual data elements mathematically to symptom elements, including symptom onset and/or severity. The correlation analysis 216 may include any data analytics technique suitable for identifying correlations in collected data sets, particularly in timeseries data sets. For example, the correlation analysis 216 may include determining one or more Pearson correlation coefficients (e.g., determining a linear correlation between two variables, a diet and/or context variable and a symptom variable, where the linear correlation produces a value between −1 and 1, where −1 indicates a perfect negative linear correlation, 1 indicates a perfect positive linear correlation, and 0 indicates no linear correlation), determining a Spearman rank correlation (e.g., determining the strength and direction of a monotonic relationship between two variables and then ranking the data to calculate a correlation based on the ranks), determining a Kendall's Tau (e.g., similar to Spearman's method, Kendall's Tau may be used to measure the strength and direction of the correlation between two variables, focusing on the number of concordant and discordant pairs of data points), determining one or more covariances (e.g., determining how two variables change together over the time-series dataset, where a positive covariance indicates that the variables tend to increase together, while a negative covariance indicates an inverse relationship), determining a distance correlation (e.g., determining linear and nonlinear dependencies between variables), performing a Principal Component Analysis (PCA) (e.g., PCA may include a dimensionality reduction technique that uncovers underlying correlations between variables by transforming the data into a new set of orthogonal variables called principal components), performing a Canonical Correlation Analysis (CCA) (e.g., CCA may identify linear relationships between two sets of variables, finding linear combinations of the variables that have the highest correlation between the two sets), performing a time series analysis with autocorrelation and/or cross-correlation techniques, employing a machine learning algorithm (e.g., with machine learning algorithms such as regression models, decision trees, and neural networks, large quantities of user data may be to uncover correlations in the data via a training process), and the like.

In an example, each data element of the timeseries symptom data may be cross-correlated with a respective data element of the timeseries context data and the timeseries diet data. In an example where the time series symptom data element (e.g., daily diarrhea severity) is represented as a function ƒ and a corresponding timeseries diet data element (e.g., dairy intake) is represented as a function g, the cross-correlation may be represented according to Eq 1.

$$( f ⋆ g ) [ n ] {=}^{△} {Σ}_{m = - ∞}^{∞} \overline{f [ m ]} g [ m + n ] \qquad Eq . 1$$

Normalizing the result of the equation for relatively small n (˜1-2 days for example) and comparing the result to a threshold and/or the corresponding result from other data elements may identify correlations between factors of diet and context with symptoms. In an example, a normalized threshold of 0.7 cross-correlation may indicate a correlation between the data elements. In an example, the normalized cross-correlation of many data elements may be considered, and the maximum of those results may be used as an identified correlation. In an example, the absolute value of the cross-correlation may be used, such that data elements in the user's that have a strong negative correlation with symptoms may be identified and used to help reinforce helpful behavior.

At this stage, the output of this correlation analysis 216 may be used for content selection 218. In an example, the content selection 218 may include a selection of content unit 222. The content selection 218 may include any process suitable for selecting an element of content (e.g., text media) based on the identified correlation (e.g., the one or more data elements correlated with a particular symptom).

In an example, the content selection 218 may determine a primary content 224 to include a human readable summary of the identified correlated data element. The primary content 224 may include one or more text templates with fields that are populated based on the results of the correlation analysis. An example, template for a primary content 224 unit may include “it seems that on days where your diarrhea is worse, you have <<if data element=diet, ‘consumed’, else ‘identified’>> more <<correlated data element>> <<for n=0, ‘that day’, else ‘in the days before.’>>” In an example, the primary content 224 may include a chart or graph indicative of the correlation. In an example, the primary content 224 may include references and/or links to specific dates within the time series data that illustrate the correlation.

In an example, the content selection 218 may determine a secondary content 226 to include a selection of content selected from a plurality of content units 228, for example. The plurality of content units 228 may each represent education material (e.g., including text media) relevant to various diet and context data elements. In an example, the plurality of content units 228 may be indexed according to their relevance to the diet and context data elements. The content selection 218 may include parsing the results of the correlation analysis to generate a query that represents the one or more correlated data elements. This query may be used on match the query terms against an index of the plurality of content units 228. In an example, the results of this matching may be ranked. For example, the ranking may include relevance, authority, previous selection (e.g., to minimize a user seeing the same content repeatedly), and the like. Then, content itself, as indicated by the ranking and index, may be retrieved from the plurality of content units 228 for delivery to the user. In an example, the query may represent a human-readable summary of the correlation (e.g., a concatenated list of the highest correlated data elements).

In an example, the content selection 218 and content delivery 220 of secondary content 226 may be performed on a schedule. For example, a predefined timeline of content delivery may be established for the user experience. In an example, where no data element has yet exceeded a correlation threshold for the user, certain default content may be selected and delivered (e.g., a general education article addressing digestive health generically). In an example, when the correlation analysis does not include any data elements exceeding a threshold, the query may be generated based on the collected user data 208. For example, the index may include data elements related to the user data 208 such as age, sex, occupation, and the like.

The digestive health content datastore 206 may include tips, small text articles formatted with figures, and/or text related to digestive health on a various set of topics. For example, it may include articles related to how certain foods affect people. It may include articles about mood affects digestive health. It may include articles about how the timing of eating may affect digestive health. It may include articles about how the spiciness of food may affect digestive health. In an example, the articles in the database may include articles that present digestive health in a more generic sense. For example, it may include articles that educate the user on diarrhea and its physiological and related information. The datastore may include an index indexing the content according to the relevant characteristics of the article. The relevant characteristics of the article may be organized according to one or more data elements. These data elements may include data elements consistent with those collected by the tracker module, such that when certain collected data elements correlate with user symptoms, those data elements are aligned with the index.

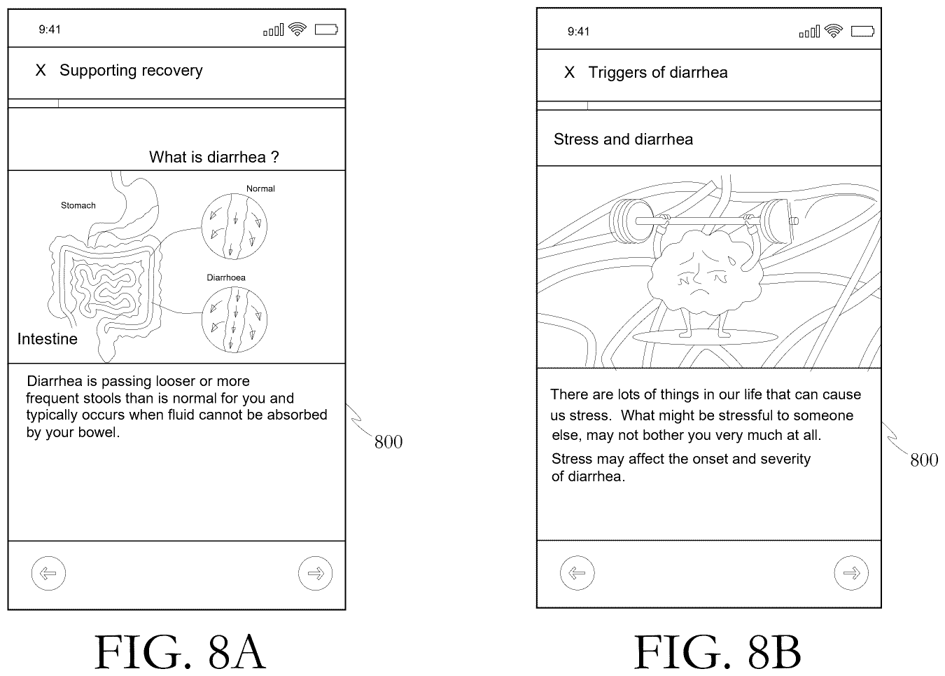

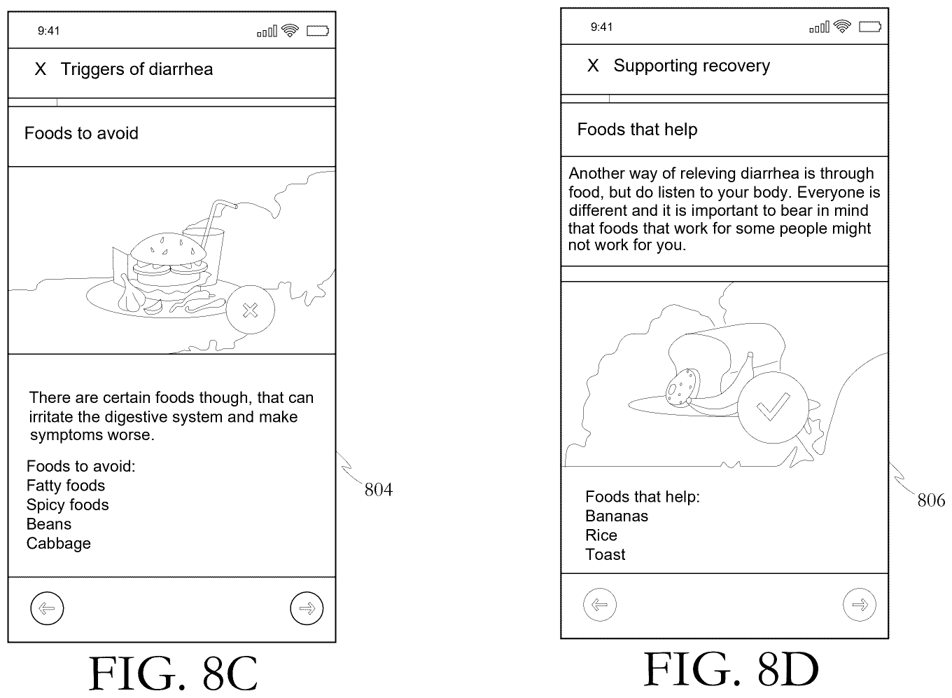

To illustrate, FIG. 8A shows an example content unit 800 that includes relatively general educational material regarding diarrhea. The content selection 218 logic when determining that a user presents with diarrhea symptoms but there is not yet a threshold correlation to another recorded data element may select such general educational material. In FIG. 8B, there is an example content unit 802 that includes an article directed to stress and its relationship to diarrhea. This content unit 802 may be indexed to “stress” and the content selection 218 logic may select this article when a threshold correlation between stress and diarrhea is found. FIGS. 8C and 8D may illustrate two articles indexed to diet items. In FIG. 8C the content unit 804 is directed to foods to avoid when suffering from diarrhea. Here, the specific food items may be included in the index, such that if any of the mentioned food items are found to be correlated with the user's onset or severity of diarrhea, this article may be selected by the content selection 218 logic. In FIG. 8D there is a content unit 806 that is directed to foods that might lessen diarrhea symptoms. Such as article may be indexed to specific food items that may be expected to be inversely correlated with symptoms (e.g., where the content selection 218 uses both positive and negative cross-correlation with symptoms). Here, generally helpful user behavior may be identified and reinforced.

Returning to FIG. 2, the content delivery 220 may include any processing (e.g., formatting) to deliver the content unit 222 to the user. For example, the content delivery 220 may include parsing of the media to deliver content properly formatted to a user's smartphone screen.

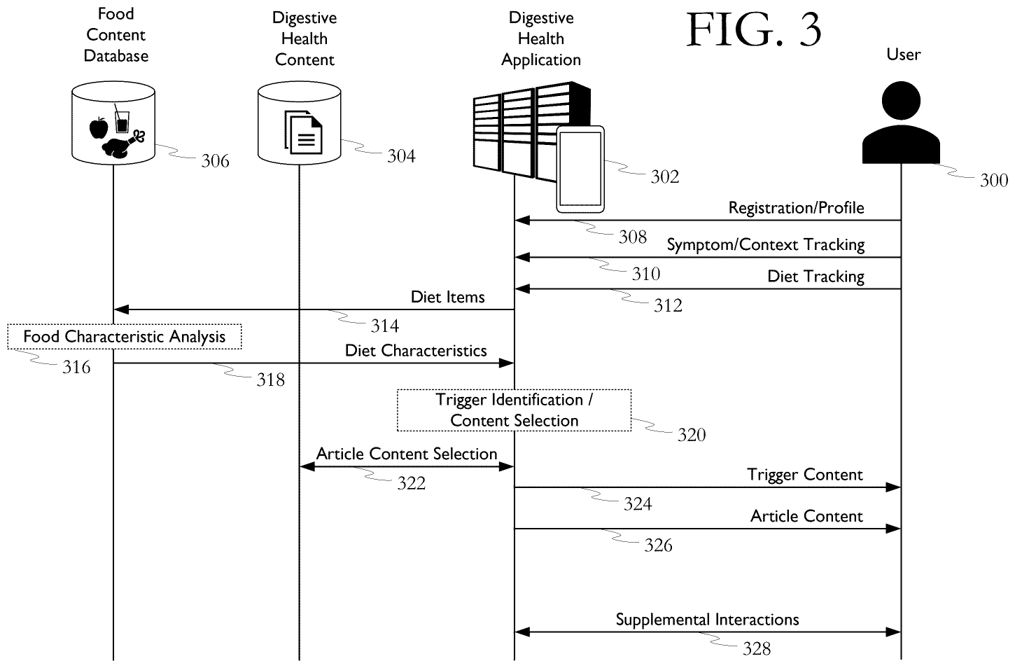

FIG. 3 is a messaging diagram of an example search engine and content delivery system. The messaging diagram illustrates interaction among a user 300, a digestive health application 302, a digestive health content datastore 304 and a food content database 306, for example. At 308, the user 300 may initially interact with the digestive health search application 302 to provide registration and/or profile information. Such registration may establish a user account with the application. Such profile information may include identification information, authentication information, and user biographical details.

At 310, the user 300 may continue interaction with the application 302 providing information (e.g., daily) in a diary interface. The information may include information about the user's symptoms (e.g., diarrhea onset and/or severity) and other factors (e.g., context) related to digestive health, for example information about the user's level of stress. This information is stored by the application 302.

At 312, the user 300 may continue interaction with the application 302 providing information (e.g., daily) in the diary interface. This information may include information about the user's diet, for example, a recording of the particular foods and drinks the user ingested at each meal throughout the day. The application 302 may receive such information and, at 314, signal this information in a query to the food content database 306. The food content database 306 may contain a mapping of common food items to respective characteristics (such as macronutrient content, for example). The food content database 306 may determine the food characteristics that correspond to the user-entered food items, at 316, and return the resulting characteristics to the application 302, at 318.

At 320, the application may identify certain triggers associated with the onset and/or severity of digestive health symptoms. For example, the application may perform a correlation analysis between the symptom data, provided by the user, and any of the data elements in the diet and/or context information from the user and/or elements from the food characteristic analysis. The results of the correlation analysis may represent triggers specific to the user's experience with digestive health symptoms (e.g., diarrhea).

At 322, the application may interact with the digestive health content datastore 304. The application may use the particularly identified triggers as the basis of a query to the datastore for selecting content to be delivered to the user. For example, if a user's stress was highly correlated with user's experience of diarrhea, the content selection, at 322, may result in the selection of content related to the relationship between stress and diarrhea, content discussing ways to reduce stress, content explaining ways to minimize symptoms when stress is unavoidable, and the like. Each such article may be selected based on an index of relevant terms corresponding to the types of data elements collected from the user and/or resolved by the food characteristic analysis, for example. And at 324, the selected content may be provided by the application to the user.

At 326, the application may provide relevant content to the user via another mechanism. Here, the application may use the trigger information to determine human-readable content associated with the trigger and its correlation itself. For example, the triggers may be used by the application to determine relevant content for the user. For example, if a user's stress was highly correlated with user's experience of diarrhea, the application may determine, at 322, content that identifies the stress trigger and its correlation to the user-presenting to the user, for example, text stating, “On days when you have indicated high levels of stress, you typically experience diarrhea.” Such content may include text and/or graphics. The application may determine this content from a set of predetermined textual elements indexed like the content discussed above. The application may determine this content dynamically, via a template with fields suitable for the communicating the correlated data elements and/or the relevant timing.

The application's technical capacity to identify triggers from the captured data and ultimately return to the user helpful, relevant content without requiring the user to perform a specific search represents a beneficial technological advance in computer information systems. With so many different and user-specific causes for digestive health symptoms, a user may not be able to identify in text the relevant terms associated with the user's experience. And traditional information systems (e.g., search engines) are not technically sufficient to provide relevant information without appropriate and specific textual input from the user. Here, the application has been configured to overcome this limitation and provide relevant content (e.g., content relevant to the user's symptom triggers and education helpful for alleviating the suffering of the user's specific experience) to the user without the user, him or herself, having to provide the appropriate and specific textual input. The delivery of content units that have been selected using such methods has been found to have reliable and significant positive impact on user health outcomes, in particular symptom severity and reported confidence in managing digestive health.

At 328, the user and the application may engage in one or more supplemental interactions. Supplemental interactions may include interaction to support additional features of the digestive health application 302.

In an example, the supplemental interactions may include support for a goal setting/tracking module of the digestive health application. Here, the application 302 may receive from the user one or more goals related to digestive health. For example, a goal may be selected from a list presented by the application 302. For example, a goal may be received in text form from the user. The goal may be subsequently presented by the application 302 to the user 300 during subsequent interactions with the application (e.g., to encourage the user and/or improve user adherence). The goal may be a measurable goal. The goal may be measurable regarding the data elements processed by the application 302. For example, the goal may be measurable regarding the symptom, context (e.g., stress and mood information), and diet information processed by the application. To illustrate, a user 300 may set a goal with the application 302 to have four or fewer diarrhea experiences per week. The application may store the user's measurable goal and may track the user's progress in view of the goal. In this illustration, the application may store the number of diarrhea experiences the user provides (at 310 for example) and may present to the user via supplemental interactions (at 328 for example) messaging related to the user's progress with the goal. Such a message, in this illustration, may state, “Congratulations, this week you have had only 2 experiences with diarrhea, which meets your goal of having four or fewer per week. Keep up the good work!”

In an example, the application may use the user-stated goal as additional information for content selection (at 320 for example). The application may present content relevant to user-stated goal. For example, the application may use keywords from the user's goal to augment the content selection process. The content selection may incorporate triggers and/or terms associated with the user's goal. Where the trigger elements are concatenated into a query, the goal terms may be appended to that concatenation, for example. The content selected and delivered to the user may relate to the correlated triggers and the user's stated goal.

In an example, the application may use the user-stated goal as the basis for the presentation of educational content (e.g., educational content presented outside of the trigger-correlation channel). For example, such content may be selected based on the user-stated goal when there is not yet an appropriate correlation, for example during the time the user has not yet entered enough data to define a correlation. For example, such content may be selected to complement and/or augment content associated with the correlation.

In an example, the supplemental interactions 328 may include one or more claim-generation interactions. Such interactions may develop supporting analytics that may be used to generate and/or support and justify certain claims to be made regarding the application. Here, the application 302 may present the user 300 one or more surveys. For example, the application 302 may present the user 300 with a survey with the registration and profiling process (at 308 for example). For example, the application 302 may present the user 300 with one or more surveys at a designated times (e.g., a set number of days since being use of the application 302), at determined time intervals (e.g., one a month), at determined usage-based intervals (e.g., after 21 user entries of diary information, such as 310 and/or 312), and the like. The user's responses to the surveys may be stored in by the application, as user profile data for example.

The surveys may relate the user's experience with digestive health, the user's experience with the application, and the like. For example, the surveys may relate to the performance of the application in affecting a user's experience with digestive health. To illustrate, based on being triggered by 90 days of tracking, the application 302 may present the user 300 with a survey to assess the user's experience—for example, asking, “Do you feel more or less in control of your journey with diarrhea?” In an example, multiple surveys may ask the same question of the user over time to determine changes.

The survey data collected from multiple users may be used for analytics. Such survey data may be stored in a database. Survey data may be anonymized. The survey data may be used to determine claims related the application 302 (e.g., claims that may be presented to the user via additional supplemental interactions 328). In this illustration, if 95% of users 300 reported feeling more in control of their journey with diarrhea, a supplemental interaction may present a message to the users stating “Keep going! 95% of users who reach 90 days of tracking report feeling more in control of their journey with diarrhea.” Such claim messages may improve user adherence and/or compliance. Such claim messages may subsequently improve the trigger correlation and content selection (e.g., via a greater volume and duration of data that accompanies greater adherence).

The supplemental interactions may include one or more milestone communications with the user. Such milestone communications may include any information from the application concerning user's progress with the application. For example, it may include information about the user's data entry practices, for example, stating to the user a fact about a positive trend or streak in data entry. For example, milestone communications may include information or a summary of any of the information entered by the user. The information may state to the user a summary of their most recent symptoms. Such milestone communications may further engage the user and improve adherence with the application. For example, milestone communication may include content units, such as those in the digestive health content datastore 304. Here, at 328, such content units may be presented to the user as general education on digestive health. Such content units may be selected at random for presentation to the user via supplemental interactions. Such content units may be pre-selected to be presented to the user over a predetermined timeline, for example. Such content units may further engage the user and increase application adherence. For example, certain content units may be presented to the user over a first number of weeks of using the application as part of a user on-boarding process. The content units may include information relevant to the use of the application and may help explain to the user how to get the greatest benefit from the application's features.

The supplemental interactions may include one or more communications or application features related to digestive health symptom management. Such interactions may include application features that would be helpful and/or beneficial to a user suffering from digestive health symptoms, such as diarrhea. For example, the supplemental interactions may include presenting the user with an interface to a public restroom geo-location database. Such a database may include a listing of public restrooms, each with its location available for searching in proximity to the user. Such a database may include a ISO (International Standards Organization) 6709 representation of the geographic point location by coordinates for each public restroom in the database. Such an interface may allow the user to comment and/or read other user's comments on each restroom entry, including helpful information regarding access, cleanliness, and location details.

The supplemental interaction may include an e-commerce interaction for products relevant to digestive health. For example, the supplemental interaction may present a user with information about over-the-counter medicines. In an example, the e-commerce interaction may include an interaction presenting the user with a mechanism to learn more about and/or obtain a product with an active ingredient of Loperamide HCl.

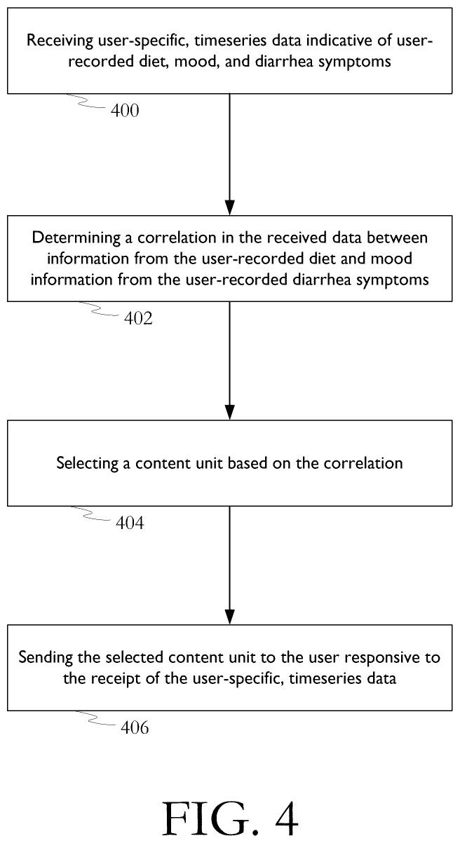

FIG. 4 is a process flow diagram illustrating an example method for selecting a content unit based on user-specific diet, mood, and diarrhea symptom data. The steps disclosed herein may be used by a computer system. For example, a computer system may include one or more processors configured to carry out the method disclosed herein. In an example, the computer system may include a smart phone application and a server application that are together configured to carry out the method disclosed herein.

At 400, user-specific, timeseries data indicative of user-recorded diet, mood, and diarrhea symptoms may be received. In this context, timeseries data refers to a plurality of data entries recorded by the user, each data entry having an associated time stamp or other time-dependent metadata. This may include receiving the data via a daily diary interface in a smartphone application. The time interval between each data entry need not be exactly or even approximately one day, for example the timeseries data may comprise multiple entries per day or two to three entries per week, and the time intervals between each pair of data entries may not be regular. For example, the timeseries data may comprise three data entries associated with three different times on a first day (e.g. Monday 9 am, 12 pm, and 7 pm diet data) and two further data entries associated with times on a second and a third day (e.g. Tuesday and Wednesday 7 am symptom data).

Timeseries data comprising data entries with multiple different time stamps may be recorded and/or received in a single instance. For example, the user may record the Monday 9 am, 12 pm, and 7 pm symptom and diet data in a single daily diary interface interaction, and timeseries data indicative of all three data entries may be received at step 400.

In an example, the user-recorded diet information may be converted to digestive health factors. For example, a food composition database may be used to convert user-recorded diet information (e.g., food items ingested) to factors relevant to digestive health (e.g., macronutrients, micronutrients, diuretic factors, capsaicin content, etc.). In an example, registration data from the user may be received from the user prior to and/or in connection with the user-specific, timeseries data.

At 402, a correlation in the received data between information from the user-recorded diet and mood information from the user-recorded diarrhea symptoms may be determined. Any correlation algorithm, suitable for timeseries data, may be used to identify relationships between the user's bouts with diarrhea and the user's preceding diet and mood. For example, the correlation may identify at least a food, factor, or a mood, that correlates with greater onset of diarrhea symptoms for the specific user. For example, the correlation may identify at least a food, factor, or a mood, that correlates with lesser onset of diarrhea symptoms for the specific user.

At 404, a content unit based on the correlation may be selected. For example, a correlation may be found in the user-specific, timeseries data that exceeds a threshold. Such a determination may trigger content selection. For example, after a pre-determined amount of data has been collected, a correlation may be identified that has the greatest correlation in the data, regardless of a threshold, and may be used in content selection.

In an example, the content unit may include a human readable summary of the user-recorded diet and mood information that represents the source of the correlation. For example, the content unit may communicate to the user the specific foods and/or moods that correlate to the diarrhea symptoms. To illustrate, such a content unit may state, “In the last two weeks, you recorded greater experiences with diarrhea following eating foods high in milk and dairy content.” Such a content unit may be selected based on a confidence of the correlation exceeding a threshold for example. Such a content unit may be implemented with one or more text-based templates in the application.

In an example, the content unit may be selected from a plurality of content units. The plurality of content units may include a private library of digestive health content, formatted for smart-phone-app-delivery. Such a selection method may include a user-specific correlation-driven search engine approach, for example, where user-specific data is used to determine a correlation from which content may be selected. In an example, each of the plurality of content units may be indexed according to at least one of symptom level, food, factor, mood, and/or timing. The content unit may be selected based on a match between the content unit's index and the identified correlation. To illustrate, a user's recorded diet and symptom data may reveal a correlation between diarrhea symptoms and previously ingested dairy food products. The data element “dairy” identified as the correlation may be used to select the content unit. For example, a content unit indexed as “dairy” may be selected for presentation to the user.

In an example, where the content unit may be selected from a plurality of content units, a human-readable summary of the correlation may be used as a search query. Here, the content units may be indexed based on their content and relevant search queries may be posed based on the conversion of the correlation to text. Such a query may incorporate more than one correlation, for example more than one data element found to correlate with the user's symptoms.

At 406, the selected content unit may be sent to the user. For example, the selected content unit may be sent to the user responsive to the receipt of the user-specific, timeseries data. In an example, the application may include certain data analytics. For example, subsequent correlation determinations and content delivery may be analyzed to assess whether use of the application and exposure to the relevant content units has affected the user's experience with digestive health symptoms. Such analysis may include generating data to show efficacy of the content delivery.

### EXAMPLE

A method according to the present invention was used to assess its effectiveness in improving health outcomes for sufferers of diarrhea and associated symptoms.

A mobile application received user-specific, timeseries data indicative of user-recorded diet, mood, and diarrhea symptoms; determined a correlation in the received data between information from the user-recorded diet and mood information from the user-recorded diarrhea symptoms; selected a content unit based on the correlation; and sent the selected content unit to the user.

Four questionnaires answered by users of the mobile application asked users to report their symptom severity across five symptoms on a points scale of 1 (least symptomatic) to 10 (most symptomatic). The questionnaires were presented every month for three months, starting with a baseline before using of the application (q0), and after one (q1), two (q2), and three (q3) months of using the application. 43 users completed q0 and q1, 25 users completed q0 and q2, and 94 users competed q0 and q3.

Table 1 shows the average number of points (recorded on a points scale between 1 and 10) that reported symptom severity decreased between q0 and q3, i.e. after three months of having been sent content selected by the application. There is clear average reduction in symptom severity across all five symptoms.

TABLE 1
Average Decrease in Symptom Severity (q0 to q3)

| Abdominal Pain | Cramps | Bloating | Wind | Nausea |
|---|---|---|---|---|
| −2.6 | −3.1 | −3.3 | −3.5 | −2.8 |

Overall, 77% of users reported a decrease in symptom severity in at least one of the recorded symptoms. Clearly, therefore, the aggregate improvements demonstrated in Table 1 are not skewed by a small subset of outlier users reporting extreme benefit. Rather, the reduction in symptom severity is an improved healthcare outcome benefitting a significant majority of users.

The most significant reductions in symptom severity were found in cramps and wind: in both cases 51% of users reported at least some decrease in symptom severity between q0 and q3.

Reduced symptom severity is, of course, an important healthcare outcome for users, and is achieved by methods according to the present invention. Also key is understanding how respondents' confidence in managing their condition change when sent relevant content units. User confidence is important because by gaining a better understanding of what triggers cause certain symptoms, even when sufferers are unable completely to avoid symptoms, they are at least able to predict when they may occur. They are then better able to go about their lives with less fear and uncertainty as to when symptoms may strike, i.e. have more confidence in managing their digestive health. In other words, greater predictability of symptom onset achieved by consuming content units leads to confidence in managing digestive health, which is associated with better mental health and wellbeing for sufferers.

Furthermore, confidence in managing digestive health can itself have a direct impact on symptoms. For sufferers triggered by anxiety and/or stress, for example, predictability and understanding of their symptoms can reduce the anxiety associated with managing their digestive health which, in turn, may reduce their symptoms.

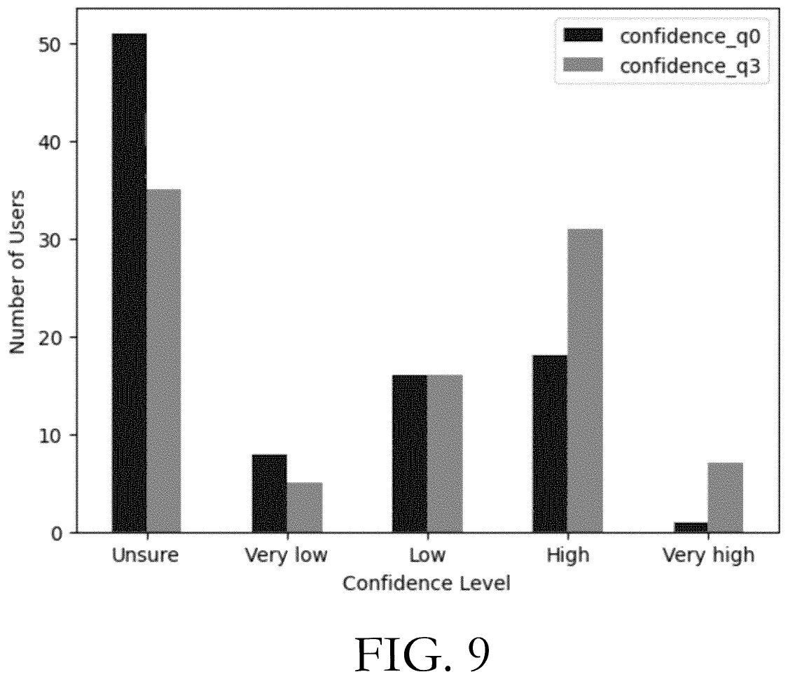

To assess the effectiveness of the mobile application in this regard, the same four questionnaires (q0 to q3) also asked users to answer the question “What is your level of confidence in managing digestive health issues?”. The results of answers to this question are shown graphically in FIG. 9.

The number of users reporting “Unsure” or “Very Low” confidence levels decreased from q0 to q3, and the number of users reporting “High” or “Very High” increased significantly from q0 to q3. Specifically, the number of “High” or “Very High” confidence users increased by more than 100%.

1. A computer-implemented method for selecting content for delivery to a user, the method comprising:
receiving user-specific, timeseries data indicative of user-recorded diet, mood, and diarrhea symptoms;
determining a correlation in the received data between information from the user-recorded diet and mood information from the user-recorded diarrhea symptoms;
selecting a content unit based on the correlation; and
sending the selected content unit to the user responsive to the receipt of the user-specific, timeseries data.

2. The method according to claim 1, further comprising converting the user-recorded diet to digestive health factors, wherein information from the user-recorded diet and mood comprises digestive health factors.

3. The method according to claim 1, wherein the correlation identifies at least a food, factor, or a mood, that correlates with greater onset of diarrhea symptoms.

4. The method according to claim 1, wherein the correlation identifies at least one of a food, a factor, a mood, or a timing that correlates with lesser onset of diarrhea symptoms.

5. The method according to claim 1, wherein the content unit is selected from a plurality of content units, wherein each of the plurality of content units is indexed according to at least one of symptom level, food, factor, mood, or timing, and wherein the content unit is selected based on a match between the content unit's index and the identified correlation.

6. The method according to claim 5, wherein the content unit is selected from the plurality of content units by using a human readable summary of the correlation as a search query.

7. The method according to claim 5, wherein the plurality of content units comprises a private library of digestive health content, formatted for smart-phone-app-delivery.

8. The method according to claim 1, wherein the content unit comprises a human readable summary of the user-recorded diet and mood information that represents the source of the correlation.

9. The method according to claim 1, further comprising receiving registration data from the user.

10. The method according to claim 1, further comprising tracking performance following delivery of the content unit and generating data to show efficacy of the content delivery.

11. The method according to claim 1, wherein content delivery is triggered based on a correlation metric exceeding a threshold.

12. The method according to claim 1, wherein the information from the user-recorded diet comprises food characteristics, as determined though a database interaction, of specific food items entered by the user.

13. The method according to claim 1, further comprising collecting a goal from the user and presenting goal tracking information based on how well the user-specific, timeseries data indicative of user-recorded diet, mood, and diarrhea symptom track to the goal.

14. The method according to claim 1, further comprising collecting a goal from the user and presenting to the user educational content based on the goal.

15. The method according to claim 1, further comprising presenting the user with an interface to a public restroom geo-location database.

16. The method according to claim 1, further comprising displaying the selected content unit on a user input-output interface.

17. A computer program comprising instructions which, when the program is executed by a computer, cause the computer to carry out the method of claim 1.

18. A computer system comprising one or more processors configured to carry out the method according to claim 1.

19. A computer system comprising a smart phone application and a server application that are together configured to carry out the method according to claim 1.
